(19) United States
(12) Patent Application Publication
(10) Pub. No.: US 2023/0096593 A1
(43) Pub. Date: Mar. 30, 2023

(54) Method and Device for Carrying Out a qPCR Method

(71) Applicant: Robert Bosch GmbH, Stuttgart (DE)

(72) Inventors: Christoph Faigle, Muensing (DE); Torsten Long, Koeln (DE)

(21) Appl. No.: 17/904,302

(22) PCT Filed: Feb. 15, 2021
(86) PCT No.: PCT/EP2021/053643

§ 371 Date: Aug. 16, 2022

(30) Foreign Application Priority Data
Feb. 25, 2020 (DE) 10 2020 202 358.1

(51) Int. Cl. C12Q 1/6851; G16B 25/20; G16B 40/10; B01L 7/00

(52) U.S. Cl. CPC C12Q 1/6851; G16B 25/20; G16B 40/10; B01L 7/52

(57) ABSTRACT

The disclosure relates to a method for operating a quantitative polymerase chain reaction (qPCR) method, having the following steps: cyclically carrying out qPCR cycles; measuring the fluorescence in each qPCR cycle in order to obtain a qPCR curve of intensity values; determining the reaction efficiency (η) for each cycle; correcting the intensity value of each cycle on the basis of the reaction efficiency (η) determined for the cycle in question in order to obtain a corrected qPCR curve; and operating the qPCR method on the basis of the corrected qPCR curve.

### TECHNICAL FIELD

The invention relates to the use of a polymerase chain reaction method (PCR method), especially for detection of the presence of a pathogen. The present invention further relates to the evaluation of qPCR measurements.

### TECHNICAL BACKGROUND

DNA strand segments in a substance to be tested, such as, for example, a serum or the like, are detected by carrying out PCR methods in automated systems. Said PCR systems make it possible to amplify and detect particular DNA strand segments to be detected, for example those which can be assigned to a pathogen. A PCR method generally comprises cyclic use of the steps of denaturation, annealing and elongation. In particular, the PCR process involves splitting of a DNA double strand into individual strands and making each of them complete again by attachment of nucleotides in order to reproduce the DNA strand segments in each cycle.

The qPCR method makes it possible to quantify the pathogen load detected using this process. To this end, at least some of the nucleotides are provided with fluorescent molecules which, upon binding to the individual strand of the DNA strand segment to be detected, activate a fluorescence property. After synthesis of the double strands, an intensity value of a fluorescence that is dependent on the number of DNA strand segments generated can be determined after each cycle.

During amplification, it is then possible to determine from the intensity values determined a qPCR curve which has a sigmoidal shape in the event of the presence of the DNA strand segment to be detected in the substance to be tested. In reality, the qPCR curves measured may contain artifacts, and so multiple parallel measurements are generally carried out in order to make a more accurate evaluation of the qPCR curves possible through averaging of the measurement values.

### DISCLOSURE OF THE INVENTION

According to the invention, a method for carrying out a qPCR method as claimed in claim 1 and a device and a qPCR system as claimed in the alternative independent claims are provided.

Further embodiments are specified in the dependent claims.

According to a first aspect, a method for conducting a quantitative polymerase chain reaction (qPCR) method is provided, comprising the following steps:
cyclic execution of qPCR cycles
measurement of the fluorescence at each qPCR cycle in order to obtain a qPCR curve composed of intensity values;
determination of a reaction efficiency for each cycle;
correction of the intensity value of each cycle depending on the reaction efficiency determined for the cycle in question in order to obtain a corrected qPCR curve;
conduction of the qPCR method depending on the shape of the corrected qPCR curve.

The qPCR method comprises cyclic repetition of the steps of denaturation, annealing and elongation. In the case of denaturation, the entire double-stranded DNA in the substance to be tested is split into two individual strands at a high temperature. In the annealing step, one of the primers added to the substance is bound to the individual strands, which primers specify the starting point of amplification of the DNA strand segments to be detected. In the elongation step, a complementary second DNA strand segment is synthesized from free nucleotides on the individual strands provided with the primer. After each of these cycles, the DNA quantity of the DNA strand segments to be detected has thus ideally doubled.

By using the qPCR method, fluorescent molecules are incorporated as labels into the DNA strand segments to be detected, and so it is possible, via measurement of the intensity of the fluorescence after each elongation step, to determine a time plot of the intensity values. The qPCR curve thus obtained comprises three distinct phases, namely a baseline, in which the intensity of the fluorescence of the fluorescent light emitted by incorporated labels is still indistinguishable from the background fluorescence, an exponential phase, in which the fluorescence intensity rises above the baseline, i.e., becomes visible, the doubling of the DNA strands in each cycle causing the fluorescence signal to exponentially rise proportional to the quantity of the DNA strand segments to be detected, and a plateau phase, in which the reagents, i.e., the primer and the free nucleotides, are no longer present in the required concentrations and no further doubling takes place.

For the detection of a specified DNA strand segment to be detected, which can correspond to a pathogen for example, the so-called ct (cycle threshold) value is relevant here. The ct value determines the start of the exponential phase and is determined by exceeding of a specific threshold, which has been defined for whichever DNA strand segment is to be detected and which is identical for all samples for the DNA strand segment to be detected, or is determined mathematically by the second derivative of the qPCR curve in the exponential phase and corresponds to the intensity value of the steepest rise of the qPCR curve. If the target value is known, the starting concentration of the DNA strand segment to be detected in the substance to be tested can be determined by back-calculation.

In reality, the qPCR curves are highly inaccurate and are subject to considerable fluctuations. Firstly, baseline drift can occur, which refers to the rise of the background fluorescence above the measurement cycles. This means that, even if no amplification is taking place, the fluorescence signal is rising. Further influencing factors which have an adverse effect on the accuracy of the qPCR curve can, for example, result from thermal noise, fluctuations or metering tolerances in the reagent concentration, and air pockets and artifacts in the fluorescence volume.

In conventional qPCR systems, what is done, firstly, is software-based correction of the PCR curves and what can be envisaged, secondly, is repeatedly measuring a sample under the same conditions and smoothing the resultant qPCR curves by averaging. However, this requires increased effort.

It is a concept of the above method to take reaction efficiency into account in each step of the PCR method, so that evaluation of the qPCR curve can be improved. The reaction efficiency is significantly determined by the reaction liquid present in the particular reaction chamber, such that there is a clear relationship between the luminescence and the measured intensity value and the quantity of the DNA strand segment to be detected. In contrast to conventional PCR methods, each reaction cycle comprising the steps of denaturation, annealing and elongation is followed by recording an image of the reaction chamber and determining therefrom a bubble volume quotient which specifies a volume fraction of an air bubble in the reaction chamber that is not contributing to the reaction with the DNA strand segment to be detected. This means that the DNA strand segments are not doubled in each PCR cycle, as would be the case in the ideal scenario; instead, they are only reproduced by a factor between 1 and 2. The reaction efficiency of this amplification corresponds to the portion of this amplification factor that goes beyond 1.

According to the above method, the reaction efficiency is thus determined after each cycle, and the respectively determined intensity value is corrected by the reaction efficiency. This yields a qPCR curve which is idealized to a reaction efficiency of 1 and which can be evaluated in a simplified manner in a subsequent step.

In the prior art, the qPCR curves are generated by combining averaged intensity values per cycle to form a curve which, after measurement, is fitted to a sigmoidal curve in order to evaluate the corrected qPCR curve, especially in order to obtain the CT value therefrom. The reaction efficiency in the standard calculation is assumed to be always the same, especially to be doubling (reaction efficiency=1). Since actual reaction efficiencies are less than 1, the sigmoidal curve thus determined is incorrect.

Furthermore, the PCR method can be carried out by conducting a reaction liquid into a reaction chamber, especially in each cycle, wherein the reaction efficiency is determined depending on an area of one or more bubbles, especially air bubbles, in the reaction chamber, especially the reaction chamber for an elongation process of the PCR process.

The reaction efficiency is impaired especially by bubbles in the reaction chamber because the quantity of the reaction mixture is reduced as a result. Since the number and size of bubbles in the reaction chamber can vary from cycle to cycle, the reduction in reaction efficiency caused thereby can likewise vary. Assuming that the formation of bubbles in the reaction chamber is the major effect leading to reduction in reaction efficiency, it is possible to define a bubble volume quotient which specifies the bubble volume as the proportion of the total volume of the reaction chamber and accordingly proportionally reduces the reaction efficiency.

It can be envisaged that an image of the reaction chamber is recorded with the aid of a camera, wherein the area of the one or more bubbles is determined with the aid of pattern recognition methods applied to the image of the reaction chamber.

Bubble detection can be carried out by known methods, such as thresholding (e.g., Otsu's method), edge detection, Hough transform, data-based methods based on neural networks and the like. Since the geometry of the reaction chamber is known, it is possible, by evaluation of a camera image of the reaction chamber, to estimate the displaced volume of reaction liquid in each cycle from the bubble extent.

In particular, the reaction efficiency can be determined with the aid of the brightness of one or more pixels of the image that correspond to the reaction liquid and with the aid of the area of the one or more bubbles as a proportion of the total area of the reaction chamber.

In one embodiment, the reaction efficiency can be determined depending on an area of one or more bubbles in the reaction chamber for at least two of the PCR subprocess steps of denaturation, annealing and elongation.

The intensity value of the remaining reaction volume can be determined by selecting only the brightness of those pixels for which it is certain that they do not belong to a bubble or to a halo (edge region) of a bubble. Alternatively, the mean of the brightness of the reaction chamber can be used, in which case the brightness can be determined taking into account a bubble volume, which generally has no fluorescence, with the aid of the bubble volume quotient. Determining which of the pixels belong to a bubble volume, which to the halo and which to the reaction liquid can be carried out with the aid of data-based methods for so-called semantic segmentation. In semantic segmentation, one class from a number of specified classes is assigned to each pixel of an image. Such a classifier makes it possible to evaluate a camera image of the reaction chamber in a simple manner.

A further possibility is to look at bubble formation in the various steps of the PCR method, namely denaturation, annealing and elongation, separately. As a result, different bubble sizes in the individual reaction steps of a PCR cycle can be taken into account, the respective bubble sizes for each of the process steps causing a reduction in reaction efficiency. Thus, camera images can be evaluated in each PCR cycle, i.e., a fluorescence measurement can be carried out at the end of denaturation, at the end of annealing, at the start of elongation and at the end of elongation. The first three values should have the same brightness if bubble volume is the same and should only differ from the intensity value of the fourth value if additional fluorescence is formed.

It is then possible to take the displaced bubble volume into account for each process step within a PCR cycle, the quotient of the intensity values of consecutive process steps being proportional to the change in reaction volume in the corresponding chamber. Assuming that the denatured and elongated DNA strand segments which are not amplified are joined together again after elongation to form the original double strands, what results after the end of a PCR cycle is a reaction efficiency value dependent on the brightness quotients between the individual process steps. Said value can then be used in correcting the recorded qPCR curve.

Furthermore, the corrected qPCR curve can be used to determine, on the basis of a classification method, whether a DNA strand segment to be detected is present or not.

In one embodiment, the qPCR method can be conducted by
signaling that a ct value is determinable,
determining the ct value from the parameterized presence function,
   if a presence of the DNA strand segment to be detected is established.

### BRIEF DESCRIPTION OF THE DRAWINGS

Embodiments will be more particularly elucidated below on the basis of the accompanying drawings, where:

FIG. 1 shows a schematic depiction of a cycle of a PCR method;

FIG. 2 shows a system for carrying out a PCR method;

FIG. 3 shows a schematic depiction of a typical qPCR curve comprising a plot of intensity values;

FIG. 4 shows a measured plot of a qPCR curve;

FIGS. 5a and 5b show ideal plots of the qPCR curve in the case of a nondetectable substance and a detectable substance, respectively; and

FIG. 6 shows a flowchart to illustrate a method for conducting a qPCR measurement;

FIG. 7 shows a photographic image of a reaction chamber containing a bubble;

FIG. 8 shows a flowchart to illustrate a further method for conducting a qPCR measurement.

### DESCRIPTION OF EMBODIMENTS

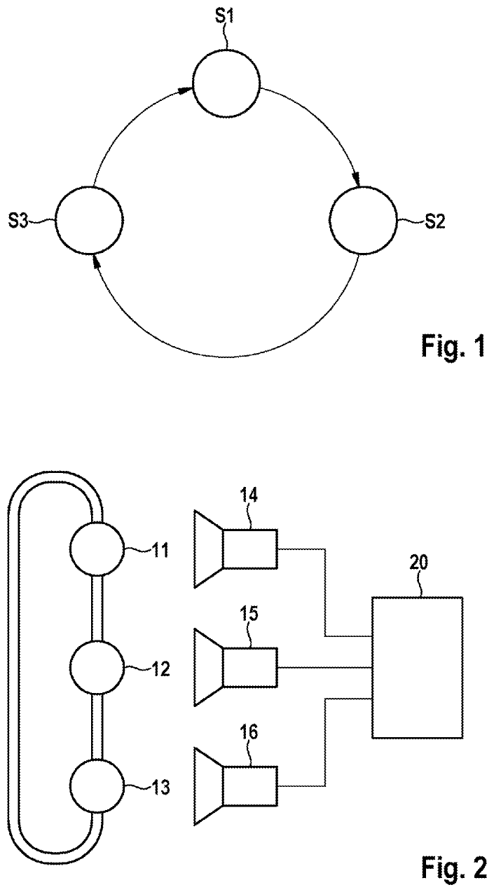

FIG. 1 shows a schematic depiction of a PCR method known per se, comprising the steps of denaturation, annealing and elongation.

In the annealing step S1, the double-stranded DNA in a substance is broken up into two individual strands at a high temperature of, for example, above 90° C. In a subsequent annealing step S2, a so-called primer is bound to the individual strands at a particular DNA position marking the start of a DNA strand segment to be detected. Said primer represents the starting point of an amplification of the DNA strand segment. In an elongation step S3, the complementary DNA strand segment is synthesized on the individual strands from free nucleotides added to the substance, starting at the position marked by the primer, with the result that the previously split individual strands have been completed to form complete double strands at the end of the elongation step.

By providing the free nucleotides or the primer with fluorescent molecules which exhibit fluorescence properties only when bound to the DNA strand segment, it is possible, by determining an intensity of a fluorescence following the elongation step S3, to obtain an intensity value through an appropriate measurement. What is assigned to the measured intensity of the fluorescent light is an intensity value.

The method comprising steps S1 to S3 is executed cyclically and the intensity values are recorded in order to obtain a plot of intensity values as a qPCR curve.

FIG. 2 depicts a system 10 for carrying out a PCR method. The system comprises three reaction chambers—the denaturation chamber 11, the annealing chamber 12 and the elongation chamber 13 for carrying out denaturation, annealing and elongation, respectively—each of which is connected to an optical system for measuring an intensity value. The optical system comprises a respective camera 14, 15, 16, which cameras are connected to a control unit 20 in which the camera images are evaluated. To this end, the reaction chambers 11, 12, 13 can be closed on at least one side with a transparent face, to which the respective camera 14, 15, 16 is directed. The cameras are used to capture a camera image of the respective reaction chamber and to provide said camera image to the control unit 20. The cameras 14, 15, 16 are suitable for detecting a fluorescent light of the PCR method. The control unit 20 is designed to carry out image processing of the recorded camera images and to determine an intensity value therefrom according to one of the methods described below.

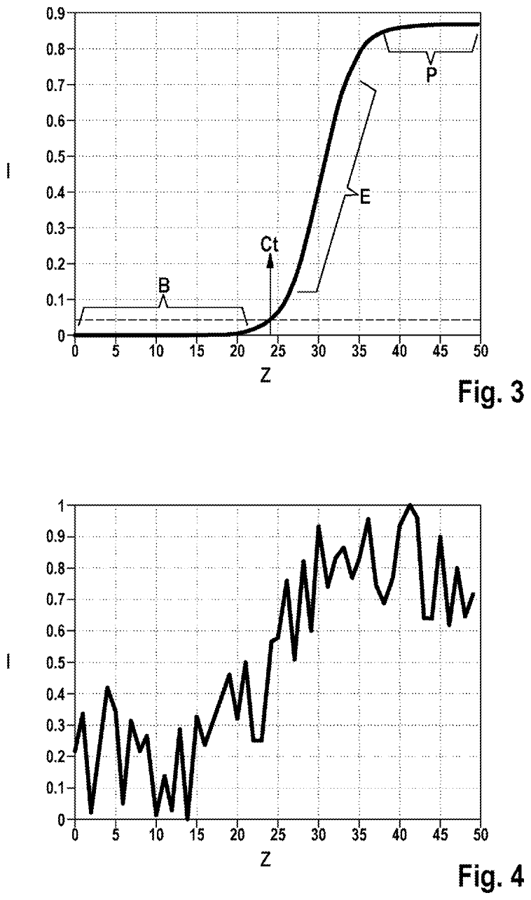

The plot of intensity values ideally has the shape depicted in FIG. 3. FIG. 3 shows a plot of normalized intensity against the cycle index z. Said plot is divided into three sections, namely a baseline section B, in which the fluorescence of the incorporated fluorescent molecules is still indistinguishable from a background fluorescence, an exponential section E, in which the intensity values are visible and rise exponentially, and in a plateau section P, in which there is flattening of the rise in intensity values, since the reagents used (solution containing nucleotides) have been consumed and no further binding to broken-up individual strands is taking place.

FIG. 4 depicts, by way of example, a plot of the intensity values obtained in a real measurement as a qPCR curve. Strong fluctuations are evident, and these may result from background fluorescence, thermal noise, fluctuations in the reagent concentrations, and bubbles and artifacts in the fluorescence volume. It is evident that it is not readily possible to determine the baseline section, exponential section and plateau section of the qPCR curve.

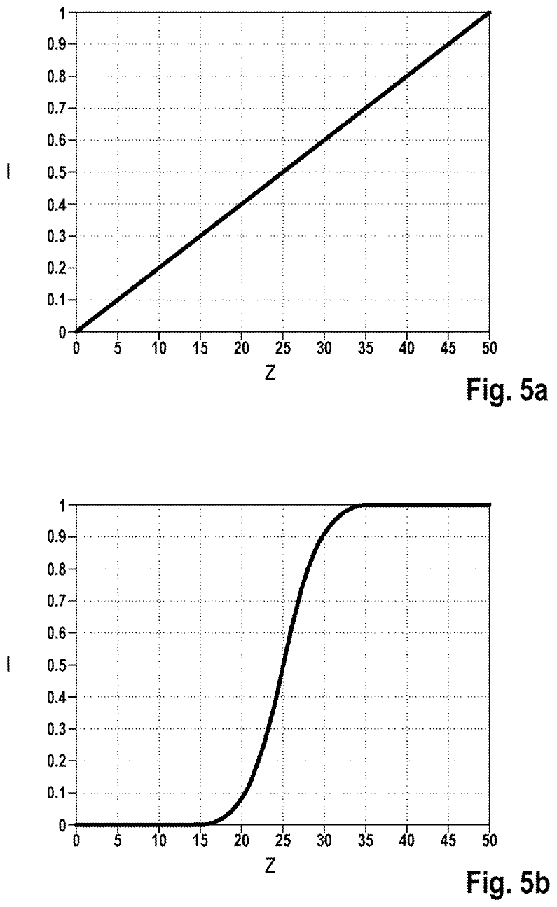

FIGS. 5a and 5b show ideal plots of a qPCR curve without the presence of a DNA strand segment to be detected and with the presence of a DNA strand segment to be detected, respectively.

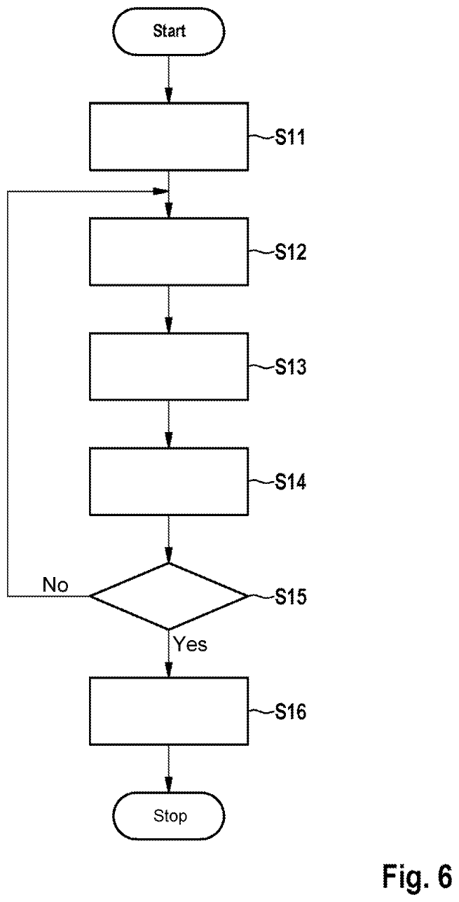

FIG. 6 depicts a flowchart to illustrate a method which is executed in the control unit. The method can be implemented in a data processing device as hardware and/or software.

In step S11, a PCR measurement method is started.

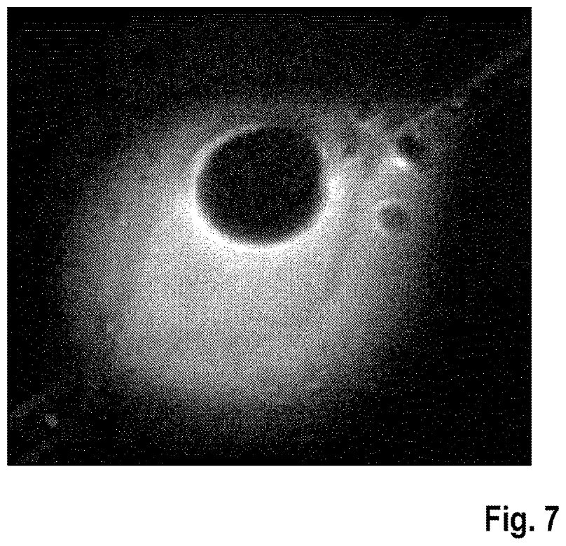

In step S12, the steps of denaturation, annealing and elongation are carried out as described above and an intensity value is determined in each cycle at the end of the elongation step. This is done by taking images of the elongation chamber 13 with the aid of the camera 16. The intensity value can be determined by taking into account bubble formation in the reaction chamber for elongation. FIG. 7 shows, by way of example, a photographic image of a reaction chamber containing an air bubble in the reaction liquid.

Methods known per se can be used to determine the presence of a bubble in the reaction chamber. Such methods, what can be performed are thresholding (Otsu's method), edge detection, Hough transform, and detection with the aid of data-based machine learning methods, for example with use of deep neural networks and the like. From the detected bubble, it is possible to determine the bubble area in the image, i.e., the area occupied by the bubble and the halo of the bubble. From the ratio between the bubble area and the total area of the reaction chamber, it is possible to determine a bubble volume quotient which specifies the volume fraction in the reaction chamber that is occupied by the bubble and hence displaces a corresponding portion of the reaction liquid. A measured intensity value is therefore only yielded by the remaining reaction liquid and can be reduced by the factor 1−V_{b} according to the volume V_{b} of the bubble, since the brightness of the fluorescence in the reaction chamber is only caused by the remaining reaction liquid.

An alternative approach can consist in selecting only the brightness of one or more pixels in the image of the reaction chamber and disregarding pixels which are part of a bubble or an associated halo. The brightnesses of pixels which can be assigned to the reaction liquid can be averaged in order to obtain a corresponding intensity value for creation of the qPCR curve. Pixels relevant to averaging can be selected by using classification methods, especially with use of machine learning methods. Said machine learning methods can be used to carry out so-called semantic segmentation. In semantic segmentation, one of a plurality of classes is assigned to each pixel of a camera image.

For this purpose, it is possible to use a data-based method comprising a classification model which has been trained with pixelwise labeled data. For this specific application, multiple n-ary classifications are conceivable:
1. Binary classification: The class “part of a bubble” or “not part of a bubble” is assigned to each pixel. Grayscale value determination is then only done via the pixels of the class “not part of a bubble” that lie within the reaction volume. This method therefore requires that the position, size and orientation of the reaction chamber be known and be unalterable relative to the camera.
2. Ternary classification: This variant adds the additional class “not part of the reaction volume” to variant 1. As a result, this method no longer requires that the position, size and orientation of the reaction chamber be known and be unalterable relative to the camera.
3. Ternary classification, second variant: This variant adds the additional class “part of the halo of a bubble” to variant 1. It may prove to be useful to evaluate the pixels in the halo of a bubble separately, since the halo can also illuminate part of the actual reaction volume. This method therefore also requires that the position, size and orientation of the reaction chamber be known and be unalterable relative to the camera.
4. Quaternary classification: This variant corresponds to a combination of variants 2 and 3. Thus, with said variant, it is possible not only to separately evaluate pixels in halos, but also to establish and compensate for changes in the relative orientation of the reaction chamber to the camera.
5. Binary classification. With this variant, the class “part of the edge of a bubble” or “not part of the edge of a bubble” is assigned to each pixel. This corresponds to edge detection. Grayscale value determination is then only done via the pixels that are completely surrounded by pixels of the class “part of the edge of a bubble”. This therefore requires that the position, size and orientation of the reaction chamber be known and be unalterable relative to the camera.

The intensity values thus obtained can be corrected in step S13 by taking reaction efficiency into account. In the prior art, the reaction efficiency is assumed to be constant, especially to be 1 in the ideal scenario. However, for actual systems, it can be assumed that the reaction efficiency varies in each cycle, meaning that the intensity values and the qPCR curve determined therefrom are incorrect.

It is assumed herein that the reaction mixture displaced by bubbles in the reaction chamber cannot contribute to amplification, since it is present in channels or other chambers, but not in the reaction chamber. The reaction efficiency therefore becomes worse.

The number of DNA strand segments per cycle under a constant reaction volume is defined as follows:

nᵢ₊₁=nᵢ(1+η)

where nᵢ corresponds to the number of DNA strand segments in cycle i and η corresponds to the chemical reaction efficiency between 0 and 1. In the simplest case, η is assumed to be 1. In a particular embodiment, this factor can also be determined by a series of experiments and thus more closely approximated.

If the displaced bubble volume V_{B,i} is then specified as the bubble volume quotient, as the quotient between the area occupied by the bubble to the total area of the reaction chamber, the result is:

nᵢ₊₁=nᵢ(1+η(1−V_{B,i}))

In the event of a presence of a bubble, the actual number of new DNA strand segments is thus below the assumed number of copied DNA strand segments. For each cycle step, it is then possible to calculate a reaction efficiency:

$${r}_{i} = \frac{{n}_{i + 1}}{{n}_{i}}$$

In step S14, the thus determined reaction efficiency in the cycle is used as a scaling factor and the qPCR curve is constructed with the aid of the corrected intensity values n_{actual, i} from the measured intensity values n_{curve, i}:

$${n}_{actual , i} = \frac{{n}_{curve , i}}{{r}_{i}}$$

In step S15, a check is made as to whether the measurement method should be terminated. For example, this may be the case after a termination criterion has been reached, such as, for example, a specified number of measurement cycles. If this is not the case (alternative: no), the method is continued in step S12, otherwise the method is ended with step S16 and the corrected qPCR curve is evaluated.

The evaluation in step S16 can be done by classification of the corrected qPCR curve with the aid of a specified classification model. This can determine whether the corrected qPCR curve indicates a presence or a nonpresence of the DNA strand segment to be detected, i.e., indicates whether the DNA strand segment to be detected is present in the substance or not.

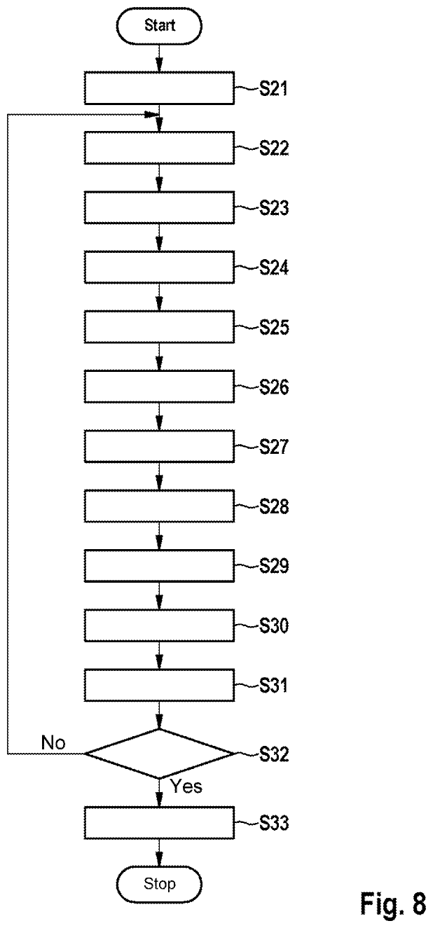

FIG. 8 depicts a flowchart to illustrate a further method which is executed in the control unit. The method can be implemented in a data processing device as hardware and/or software.

In step S21, the qPCR method is started.

In step S22, the denaturation step S1 is carried out in the corresponding denaturation reaction chamber.

In step S23, a brightness h_{D} of the fluorescent light is recorded at the end of denaturation by the corresponding camera 14 of the denaturation chamber 11.

In step S24, the annealing process of step S2 is started.

In step S25, a brightness h_{A} of the fluorescent light is recorded at the end of the annealing process by the corresponding camera 15 of the annealing chamber 12.

In step S26, the reaction liquid is conducted into the elongation chamber 13.

In step S27, a brightness h_{E,A} of the fluorescent light is recorded at the start of the elongation process by the corresponding camera 16 of the elongation chamber 13.

In step S28, the elongation process is started.

In step S29, a brightness h_{e,e,i} of the fluorescent light is recorded at the end of the elongation step by the corresponding camera 16 of the elongation chamber 13.

Theoretically, the values h_{d,i}, h_{a,i}, h_{e,a,i} have the same brightness, since no amplification is taking place, and only differ from the brightness value h_{e,e,i} if additional fluorescence results from the amplification of the elongation process.

In what follows, a total reaction efficiency is calculated in step S30.

Owing to bubble formation differing in bubble size, the brightnesses in the various reaction chambers 11, 12, 13 can vary, however. This variation can be used as an indicator for whether bubbles have formed and to what extent. Owing to bubble formation, the reaction liquid is displaced and the efficiency of the individual substeps is thus reduced, since the entire reaction liquid is not available for the reaction. Therefore, bubble volume quotients q as quotients of the brightnesses between the individual substeps can be determined as follows:

$${q}_{d , i + 1} = \frac{{h}_{d , i + 1}}{{h}_{e , e , i}} \\ {q}_{a , i} = \frac{{h}_{a , i}}{{h}_{d , i}} \\ {q}_{e , i} = \frac{{h}_{e , a , i}}{{h}_{a , i}} \\ η = \frac{{h}_{e , e , i}}{{h}_{e , a , i}}$$

If one of the quotients q should be greater than 1, it is set to 1, since the reaction liquid not processed in the previous step cannot be further processed in the next substep.

What therefore arises for each substep is the quantity of processed DNA strand segments (n_{d,i} for the number of DNA strand segments after the denaturation in the i-th cycle, n_{a,i} for the number of DNA strand segments after the annealing in the i-th cycle, n_{e,i} for the number of DNA strand segments after the elongation in the i-th cycle)

n_{d,i}=nᵢ·q_{d,i}

n_{a,i}=nᵢ·q_{a,i}

n_{e,i}=nᵢ·q_{e,i}

The number of DNA strand segments available for elongation and incorporation of fluorescence as at the start of the last substep therefore corresponds to

n_{e,a,i}=nᵢ·q_{d,i}·q_{a,i}·q_{e,i}

Assuming that denatured and elongated DNA strand segments which are not amplified are joined together again to form the original double strands, what results after the PCR cycle, i.e., at the end of the elongation phase, is

nᵢ₊₁=nᵢ(1+η·q_{d,i}·q_{a,i}·q_{e,i})

as the quantity of DNA strand segments present in total in the reaction liquid.

In the event of the presence of a bubble in the reaction chamber, the actual number of new DNA strand segments is thus below the theoretically assumed number of DNA strand segments.

For each cycle step, it is then possible to calculate a total reaction efficiency

$${r}_{i} = \frac{{n}_{i + 1}}{{n}_{i}}$$

This reaction efficiency can, as in the exemplary embodiment of FIG. 6, be used as a scaling factor for the measured intensity value at the end of the elongation phase.

In step S31, the thus determined reaction efficiency in the cycle is used as a scaling factor and the qPCR curve is constructed with the aid of the corrected intensity values n_{actual,i} from the measured intensity values n_{curve,i}:

$${n}_{actual , i} = \frac{{n}_{curve , i}}{{r}_{i}}$$

In step S32, a check is made as to whether the measurement method should be terminated. For example, this may the case after a termination criterion has been reached, such as, for example, a specified number of measurement cycles. If this is not the case (alternative: no), the method is continued in step S22, otherwise the method is ended with step S33 and the corrected qPCR curve is evaluated.

The evaluation in step S33 can be done by classification of the corrected qPCR curve with the aid of a specified classification model. This can determine whether the corrected qPCR curve indicates a presence or a nonpresence of the DNA strand segment to be detected, i.e., indicates whether the DNA strand segment to be detected is present in the substance or not.

1. A method for conducting a quantitative polymerase chain reaction (qPCR) process the method comprising:
cyclically executing qPCR cycles;
measuring a fluorescence at each qPCR cycle to obtain a qPCR curve composed of intensity values;
determining a reaction efficiency for each qPCR cycle;
correcting a respective intensity value of each respective qPCR cycle depending on the reaction efficiency determined for the respective qPCR cycle to obtain a corrected qPCR curve; and
conducting the qPCR process depending on a shape of the corrected qPCR curve.

2. The method as claimed in claim 1 further comprising:
conducting a reaction liquid into a reaction chamber,
wherein the reaction efficiency is determined depending on an area of at least one bubble in the reaction chamber.

3. The method as claimed in claim 2 further comprising:
recording an image of the reaction chamber with a camera; and
determining the area of the at least one bubble with pattern recognition methods applied to the image of the reaction chamber.

4. The method as claimed in claim 3, the determining further comprising:
determining the reaction efficiency with a brightness of at least one pixel of the image that corresponds to the reaction liquid and the area of the at least one bubble as a proportion of a total area of the reaction chamber.

5. The method as claimed in claim 1, the determining further comprising:
determining the reaction efficiency depending on an area of at least one bubble in a reaction chamber for at least two of (i) a denaturation process, (ii) an annealing and (iii) a elongation process.

6. The method as claimed in claim 1 further comprising:
determining, using the corrected qPCR curve, based on a classification method, whether a DNA strand segment to be detected is present.

7. The method as claimed in claim 1, the conducting the qPCR process further comprising:
signaling that a ct value is determinable; and
determining the ct value from a parameterized presence function in response to a presence of the DNA strand segment to be detected being established.

8. A device for conducting a quantitative polymerase chain reaction (qPCR) process, the device being configured to:
cyclically execute qPCR cycles;
measure a fluorescence at each qPCR cycle to obtain a qPCR curve composed of intensity values;
determine a reaction efficiency for each qPCR cycle;
correct a respective intensity value of each respective qPCR cycle depending on the reaction efficiency determined for the respective qPCR cycle to obtain a corrected qPCR curve; and
conduct the qPCR process depending on a shape of the corrected qPCR curve.

9. The method as claimed in claim 1, wherein the method is carried out by executing a computer program.

10. A non-transitory electronic storage medium storing a computer program for conducting a quantitative polymerase chain reaction (qPCR) process, the computer program being configured to, when executed by a computer, cause the computer to:
cyclically execute of qPCR cycles;
measure a fluorescence at each qPCR cycle to obtain a qPCR curve composed of intensity values;
determine a reaction efficiency for each qPCR cycle;
correct a respective intensity value of each respective qPCR cycle depending on the reaction efficiency determined for the respective qPCR cycle to obtain a corrected qPCR curve; and
conduct the qPCR process depending on a shape of the corrected qPCR curve.

11. The method as claimed in claim 2, the conducting the reaction liquid further comprising:
conducting a reaction liquid into a reaction chamber in each qPCR cycle.

12. The method as claimed in claim 2, wherein the reaction efficiency is determined depending on an area of at least one bubble in the reaction chamber for an elongation process.
